(19) United States
(12) Patent Application Publication
(10) Pub. No.: US 2015/0004601 A1
(43) Pub. Date: Jan. 1, 2015

(54) MASSIVELY PARALLEL SEQUENCING OF RANDOM DNA FRAGMENTS FOR DETERMINATION OF FETAL FRACTION

(71) Applicant: Ariosa Diagnostics, Inc., San Jose, CA (US)

(72) Inventors: Craig Struble, San Jose, CA (US); Eric Wang, San Jose, CA (US); Arnold Oliphant, San Jose, CA (US)

(21) Appl. No.: 14/299,963

(22) Filed: Jun. 9, 2014

Related U.S. Application Data
(60) Provisional application No. 61/840,769, filed on Jun. 28, 2013.

(51) Int. Cl. C12Q 1/68

(52) U.S. Cl. CPC C12Q 1/6869
USPC 435/6.11

(57) ABSTRACT

The present invention provides methods for determining the fraction of fetal DNA in a maternal sample using massively parallel shotgun sequencing techniques and statistical probability calculations. The invention utilizes a novel method of identifying polymorphisms through the sequencing process that align to designated regions in the genome. By identifying a statistically significant number of such polymorphisms in multiple designated regions across the genome the fetal fraction, or estimation thereof, can be determined. In certain aspects, the observed distribution of polymorphisms in the genome of a maternal sample can be compared to a fetal proportion reference to estimate the fetal fraction in the sample.

### CROSS-REFERENCE TO RELATED APPLICATIONS

This application claims the benefit of U.S. Provisional Patent Application Ser. No. 61/840,769, filed Jun. 28, 2013 and is incorporated herein by reference.

### FIELD OF THE INVENTION

This invention relates to the determination of genetic variation and fetal fraction in maternal samples using massively parallel sequencing of random DNA fragments.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions. Recent advances in diagnostics have focused on less invasive mechanisms for determining disease risk, presence and prognosis. Diagnostic processes for determining genetic anomalies have become standard techniques for identifying specific diseases and disorders, as well as providing valuable information on disease source and treatment options.

The identification of cell free nucleic acids in biological samples such as blood and plasma allow less invasive techniques such as blood extraction to be used in making clinical decisions. For example, cell free DNA from malignant solid tumors has been found in the peripheral blood of cancer patients; individuals who have undergone transplantation have cell free DNA from the transplanted organ present in their bloodstream; and cell-free fetal DNA and RNA have been found in the blood and plasma of pregnant women. In addition, detection of nucleic acids from infectious organisms, such as detection of viral load or genetic identification of specific strains of a viral or bacterial pathogen, provides important diagnostic and prognostic indicators. Cell free nucleic acids from a source separate from the patient's own normal cells can thus provide important medical information, e.g., about treatment options, diagnosis, prognosis and the like.

The sensitivity of such testing is often dependent upon the identification of the amount of nucleic acid from the different sources, and in particular identification of a low level of nucleic acid from one source in the background of a higher level of nucleic acids from a second source. Detecting the contribution of the minor nucleic acid species to cell free nucleic acids present in the biological sample can provide accurate statistical interpretation of the resulting data.

There is thus a need for processes for calculating copy number variation (CNV) in one or more genomic regions in a biological sample using information on contribution of nucleic acids in the sample. The present invention addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present invention provides methods for determining the fraction of fetal DNA in a maternal sample using massively parallel shotgun sequencing techniques and statistical probability calculations. The invention utilizes a novel method of identifying polymorphisms that align to designated regions in the genome via the massively parallel sequencing techniques. By identifying a statistically significant number of such polymorphisms in multiple designated regions across the genome the fetal fraction, or an estimation thereof, can be determined.

In a preferred aspect, the polymorphisms used are single nucleotide polymorphisms (“SNPs”), and the SNPs are biallelic across populations, i.e., only two bases (alleles) are observed across the general populations at such SNP sites. In certain aspects, the SNPs used are selected to be biallelic for a particular population (e.g. a geographic population) from which the maternal sample is obtained. In certain embodiments, SNPs used in the present invention include any SNP identified through sequencing and detection processes. In other certain embodiments, SNPs used in the analysis are informative SNPs, including but not limited to tag SNPs.

Thus, in one embodiment, the invention provides a method for determining fetal fraction in a maternal sample, wherein the method comprises obtaining a mixture of fetal and maternal cell-free DNA from said maternal sample, conducting massively parallel DNA sequencing of random DNA fragments from the mixture of fetal and maternal genomic DNA to determine the sequence of said DNA fragments; identifying nucleic acids corresponding to a plurality of informative SNPs in designated regions of the genomic DNA by alignment of the sequenced DNA fragments to a reference, determining the relative frequency of the sequenced informative SNPs, and calculating the fetal fraction of the maternal sample using the relative frequency of the sequenced informative single nucleotide polymorphisms.

The sequence obtained from the random DNA fragments is from about 15 bp to about 150 bp in length, more preferably from about 25 bp to about 100 bp in length.

The genomic DNA used from the maternal sample is preferably cell-free DNA, such as cell-free DNA from maternal plasma or serum.

The accuracy of the calculation of fetal fraction is dependent upon the number of informative SNPs (including tag SNPs) utilized in the calculation and the distribution of the SNPs in the different regions of the genome. Thus, the methods preferably further comprise determining the number of SNPs and/or tag SNPs necessary for a statistically significant estimation of fetal fraction in the maternal sample.

The number of SNPs required to make a statistically significant estimation of fetal fraction also depends on the level of multiplexing of samples in the sequencing process itself. For example, the number of informative SNPs required to determine fetal fraction in samples multiplexed one hundred-fold in the sequencing process is on the order of 10 times greater than the number of informative SNPs required to determine fetal fraction in samples multiplexed fifty-fold in the sequencing process.

Thus, in some embodiments the methods involve determination of fetal fraction in five or more maternal samples sequenced simultaneously. This method comprises obtaining a mixture of fetal and maternal cell-free DNA from each maternal sample, conducting massively parallel DNA sequencing of random DNA fragments from the mixture of fetal and maternal genomic DNA of each maternal sample to determine the sequence of said DNA fragments; identifying nucleic acids corresponding to a plurality of informative SNPs in designated regions of the genomic DNA by alignment of the sequenced DNA fragments of each sample to a reference, identifying the number of informative SNPs necessary to obtain a statistically significant estimation of fetal fraction in each of the maternal samples; determining the relative frequency of at least the identified number of sequenced informative SNPs in each sample, and calculating the fetal fraction of the maternal samples using the relative frequency of the sequenced informative single nucleotide polymorphisms.

Preferably, the fetal fraction is determined in ten or more maternal samples sequenced simultaneously, preferably twenty or more maternal samples sequenced simultaneously, more preferably fifty or more maternal samples sequenced simultaneously, or even more preferably ninety or more maternal samples sequenced simultaneously.

In certain embodiments, the informative SNPs used to determine fetal fraction are tag SNPs. The invention thus also provides a method for determining fetal fraction in a maternal sample, wherein the method comprises obtaining a mixture of fetal and maternal genomic DNA from said maternal sample, conducting massively parallel DNA sequencing of random DNA fragments from the mixture of fetal and maternal genomic DNA to determine the sequence of said DNA fragments, identifying nucleic acids corresponding to a plurality of tag SNPs by alignment of the sequenced DNA fragments to a reference, determining the relative frequency of the sequenced tag SNPs, and calculating the fetal fraction of the maternal sample using the relative frequency of the sequenced tag SNPs.

The invention also provides methods for simultaneously determining the presence or absence of a fetal aneuploidy and fetal fraction in a maternal sample comprising: obtaining a mixture of fetal and maternal genomic DNA from a maternal sample, conducting massively parallel DNA sequencing of random DNA fragments from the mixture of fetal and maternal genomic DNA to determine the sequence of said DNA fragments, aligning the DNA fragment sequences generated from step b) to a reference; determining a relative frequency of DNA fragment sequences corresponding to a plurality of informative single nucleotide polymorphisms based on the alignment of the DNA fragment sequences to the reference, determining a relative frequency of DNA fragment sequences from a first chromosome based on the alignment of the DNA fragment sequences to the reference, determining a relative frequency of DNA fragment sequences from a second chromosome based on the alignment of the DNA fragment sequences to the reference, and determining the fetal fraction of the maternal sample and the presence or absence of a fetal aneuploidy using the relative frequency of the sequenced informative single nucleotide polymorphisms and the relative frequencies of DNA fragment sequences from the first and second chromosome.

The invention also provides methods for statistically determining the likelihood of a fetal chromosomal abnormality in a maternal sample comprising fetal and maternal cell-free genomic DNA, the method comprising: obtaining a mixture of fetal and maternal genomic DNA from a maternal sample; conducting massively parallel DNA sequencing of random DNA fragments from the mixture of fetal and maternal genomic DNA to determine the sequence of said DNA fragments; aligning the generated DNA fragment sequences to a reference; determining a relative frequency of DNA fragment sequences corresponding to a plurality of informative single nucleotide polymorphisms based on the alignment of the DNA fragment sequences to the reference; determining a relative frequency of DNA fragment sequences from a first chromosome based on the alignment of the DNA fragment sequences to the reference; determining a relative frequency of DNA fragment sequences from a second chromosome based on the alignment of the DNA fragment sequences to the reference; determining the fetal fraction of the maternal sample using the relative frequency of the sequenced informative single nucleotide polymorphisms; and statistically determining the likelihood of a fetal chromosomal abnormality based on the relative frequencies of DNA fragment sequences from the first and second chromosome.

In yet another aspect, the invention provides methods for estimating fetal fraction in a maternal sample, wherein the method comprises: obtaining a mixture of fetal and maternal genomic DNA from said maternal sample; conducting massively parallel DNA sequencing of random DNA fragments from the mixture of fetal and maternal genomic DNA of step a) to determine the sequence of said DNA fragments; identifying nucleic acids corresponding to a plurality of single nucleotide polymorphisms by alignment of the sequenced DNA fragments to a reference; determining the relative frequency of the sequenced single nucleotide polymorphisms; comparing the determined relative frequencies of the single nucleotide polymorphisms to a fetal proportion reference; and estimating the fetal fraction of the maternal sample based on the comparison of the determined relative frequencies of the single nucleotide polymorphisms to the fetal proportion reference.

The fetal proportion reference can be either based on empirical information or simulated information. The fetal fraction in a maternal sample is estimated by comparison of the observed distribution of SNPs in a sample to a fetal proportion reference, and preferably a fetal proportion reference based on simulated distributions. The distribution of the fetal proportion reference most closely matching the observed distribution provide an estimate of the fetal fraction.

The fetal aneuploidy can be any full or partial aneuploidy. Preferably an aneuploidy detected is chromosome 13, chromosome 18, chromosome 21, chromosome X or chromosome Y.

These and other aspects, features and advantages will be provided in more detail as described herein.

### BRIEF DESCRIPTION OF THE FIGURES

FIG. 1 is a simplified flow chart of the general steps utilized in certain embodiments of the invention.

FIG. 2 is a simplified flow chart of the general steps utilized in certain embodiments of the invention.

FIG. 3 is a graphic illustration of a fetal proportion reference. Distributions are determined for each fetal fraction based on simulated data. The X axis represents the number of obtained sequence reads of a single allele at a biallelic locus. The Y axis represents the fraction of fragments analyzed from an MPSS analysis expected to contain each SNP.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “amplified nucleic acid” is any nucleic acid molecule whose amount has been increased at least two fold by any nucleic acid amplification or replication method performed in vitro as compared to its starting amount in a mixed sample.

The term “chromosomal abnormality” refers to any genetic variation that affects all or part of a chromosome equal to or greater than a single locus. The genetic variants may include but not be limited to any CNV such as duplications or deletions, translocations, inversions, and mutations. Examples of chromosomal abnormalities include, but are not limited to, Down Syndrome (Trisomy 21), Edwards Syndrome (Trisomy 18), Patau Syndrome (Trisomy 13), Klinefelter's Syndrome (XXY), Triple X syndrome, XYY syndrome, Trisomy 8, Trisomy 16, Turner Syndrome, Robertsonian translocation, DiGeorge Syndrome and Wolf-Hirschhorn Syndrome.

The term “copy number variation” or “CNV” as used interchangeably herein are alterations of the DNA of a genome that results in a cell having an abnormal number of copies of one or more loci in the DNA. CNVs that are clinically relevant can be limited to a single gene or include a contiguous set of genes. A CNV can also correspond to relatively large regions of the genome that have been deleted, inverted or duplicated on certain chromosomes, up to an including one or more additional copies of a complete chromosome. The term CNV as used herein does not refer to any sequence-related information, but rather to quantity or “counts” of genetic regions present in a sample.

The term “diagnostic tool” as used herein refers to any composition or assay of the invention used in combination as, for example, in a system in order to carry out a diagnostic test or assay on a patient sample.

The term “disease trait” refers to a monogenic or polygenic trait associated with a pathological condition, e.g., a disease, disorder, syndrome or predisposition.

The term “fetal proportion reference” refers to a set of single nucleotide polymorphism distributions that is used in certain embodiments as a reference to compare observed distributions of one or more maternal samples to evaluate the fetal proportion of the maternal sample. The fetal proportion reference may be provided as a calculation, a graphical representation, or other comparator that provides a statistical difference in SNP identification based on the fetal fraction of a maternal sample. The fetal proportion reference may be based on empirical or simulated information.

The term “hybridization” generally means the reaction by which the pairing of complementary strands of nucleic acid occurs. DNA is usually double-stranded, and when the strands are separated they will re-hybridize under the appropriate conditions. Hybrids can form between DNA-DNA, DNA-RNA or RNA-RNA. They can form between a short strand and a long strand containing a region complementary to the short one. Imperfect hybrids can also form, but the more imperfect they are, the less stable they will be (and the less likely to form).

The term “informative locus” as used herein refers to a locus that can be used to distinguish DNA from a first source (e.g., a major source) from DNA from a second source (e.g., a minor source) in a sample. Informative loci may include polymorphisms such as informative SNPs, including but not limited to tag SNPs.

The terms “locus” and “loci” as used herein refer to a region of known location in a genome.

The term “major source” refers to a source of nucleic acids in a sample from an individual that is representative of the predominant genomic material in that individual.

The term “maternal sample” as used herein refers to any sample taken from a pregnant mammal which comprises both fetal and maternal cell free genomic material (e.g., DNA). Preferably, maternal samples for use in the invention are obtained through relatively non-invasive means, e.g., phlebotomy or other standard techniques for extracting peripheral samples from a subject.

The term “minor source” refers to a source of nucleic acids within an individual that is present in limited amounts and which is distinguishable from the major source due to differences in its genomic makeup and/or expression. Examples of minor sources include, but are not limited to, fetal cells in a pregnant female, cancerous cells in a patient with a malignancy, cells from a donor organ in a transplant patient, nucleic acids from an infectious organism in an infected host, and the like.

The term “mixed sample” as used herein refers to any sample comprising cell free genomic material (e.g., DNA) from two or more cell types of interest, one being a major source and the other being a minor source within a single individual. Mixed samples include samples with genomic material from both a major and a minor source in an individual, which may be e.g., normal and atypical somatic cells, or cells that comprise genomes from two different individuals, e.g., a sample with both maternal and fetal genomic material or a sample from a transplant patient that comprises cells from both the donor and recipient. Mixed samples are preferably peripherally derived, e.g., from blood, plasma, serum, etc.

The term “monogenic trait” as used herein refers to any trait, normal or pathological, that is associated with a mutation or polymorphism in a single gene. Such traits include traits associated with a disease, disorder, or predisposition caused by a dysfunction in a single gene. Traits also include non-pathological characteristics (e.g., presence or absence of cell surface molecules on a specific cell type).

The term “non-maternal” allele means an allele with a polymorphism and/or mutation that is found in a fetal allele (e.g., an allele with a de novo SNP or mutation) and/or a paternal allele, but which is not found in the maternal allele.

By “non-polymorphic”, when used with respect to detection of selected loci, is meant a detection of such locus, which may contain one or more polymorphisms, but in which the detection is not reliant on detection of the specific polymorphism within the region. Thus a selected locus may contain a polymorphism, but detection of the region using the assay system of the invention is based on occurrence of the region rather than the presence or absence of a particular polymorphism in that region.

As used herein “nucleotide” refers to a base-sugar-phosphate combination. Nucleotides are monomeric units of a nucleic acid sequence (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP.

According to the present invention, a “nucleotide” may be unlabeled or detectably labeled by well known techniques. Fluorescent labels and their attachment to oligonucleotides are described in many reviews, including Haugland, Handbook of Fluorescent Probes and Research Chemicals, 9th Ed., Molecular Probes, Inc., Eugene Oreg. (2002); Keller and Manak, DNA Probes, 2nd Ed., Stockton Press, New York (1993); Eckstein, Ed., Oligonucleotides and Analogues: A Practical Approach, IRL Press, Oxford (1991); Wetmur, Critical Reviews in Biochemistry and Molecular Biology, 26:227-259 (1991); and the like. Other methodologies applicable to the invention are disclosed in the following sample of references: Fung et al., U.S. Pat. No. 4,757,141; Hobbs, Jr., et al., U.S. Pat. No. 5,151,507; Cruickshank, U.S. Pat. No. 5,091,519; Menchen et al., U.S. Pat. No. 5,188,934; Begot et al., U.S. Pat. No. 5,366,860; Lee et al., U.S. Pat. No. 5,847,162; Khanna et al., U.S. Pat. No. 4,318,846; Lee et al., U.S. Pat. No. 5,800,996; Lee et al., U.S. Pat. No. 5,066,580: Mathies et al., U.S. Pat. No. 5,688,648; and the like. Labeling can also be carried out with quantum dots, as disclosed in the following patents and patent publications: U.S. Pat. Nos. 6,322,901; 6,576,291; 6,423,551; 6,251,303; 6,319,426; 6,426,513; 6,444,143; 5,990,479; 6,207,392; 2002/0045045; and 2003/0017264. Detectable labels include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo)benzoic acid (DABCYL), CASCADE BLUE® (pyrenyloxytrisulfonic acid), OREGON GREEN™ (2′,7′-difluorofluorescein), TEXAS RED™ (sulforhodamine 101 acid chloride), Cyanine and 5-(2′-aminoethyl)aminonaphthalene-1-sulfonic acid (EDANS). Specific examples of fluroescently labeled nucleotides include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink FluorX-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, CASCADE BLUE®-7-UTP (pyrenyloxytrisulfonic acid-7-UTP), CASCADE BLUE®-7-dUTP (pyrenyloxytrisulfonic acid-7-dUTP), fluorescein-12-UTP, fluorescein-12-dUTP, OREGON GREEN™ 488-5-dUTP (2′,7′-difluorofluorescein-5-dUTP), RHODAMINE GREEN™-5-UTP ((5-{2-[4-(aminomethyl)phenyl]-5-(pyridin-4-yl)-1H-i-5-UTP)), RHODAMINE GREEN™-5-dUTP ((5-{2-[4-(aminomethyl)phenyl]-5-(pyridin-4-yl)-1H-i-5-dUTP)), tetramethylrhodamine-6-UTP, tetramethylrhodamine-6-dUTP, TEXAS RED™-5-UTP (sulforhodamine 101 acid chloride-5-UTP), TEXAS RED™-5-dUTP (sulforhodamine 101 acid chloride-5-dUTP), and TEXAS RED™-12-dUTP (sulforhodamine 101 acid chloride-12-dUTP) available from Molecular Probes, Eugene, Oreg. The terms “oligonucleotides” or “oligos” as used herein refer to linear oligomers of natural or modified nucleic acid monomers, including deoxyribonucleotides, ribonucleotides, anomeric forms thereof, peptide nucleic acid monomers (PNAs), locked nucleotide acid monomers (LNA), and the like, or a combination thereof, capable of specifically binding to a single-stranded polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g., 8-12, to several tens of monomeric units, e.g., 100-200 or more. Suitable nucleic acid molecules may be prepared by the phosphoramidite method described by Beaucage and Carruthers (Tetrahedron Lett., 22:1859-1862 (1981)), or by the triester method according to Matteucci, et al. (J. Am. Chem. Soc., 103:3185 (1981)), both incorporated herein by reference, or by other chemical methods such as using a commercial automated oligonucleotide synthesizer.

The term “polygenic trait” as used herein refers to any trait, normal or pathological, that is associated with a mutation or polymorphism in more than a single gene. Such traits include traits associated with a disease, disorder, syndrome or predisposition caused by a dysfunction in two or more genes. Traits also include non-pathological characteristics associated with the interaction of two or more genes.

As used herein the term “polymerase” refers to an enzyme that links individual nucleotides together into a long strand, using another strand as a template. There are two general types of polymerase—DNA polymerases, which synthesize DNA, and RNA polymerases, which synthesize RNA. Within these two classes, there are numerous sub-types of polymerases, depending on what type of nucleic acid can function as template and what type of nucleic acid is formed.

As used herein “polymerase chain reaction” or “PCR” refers to a technique for amplifying a specific piece of selected DNA in vitro, even in the presence of excess non-specific DNA. Primers are added to the selected DNA, where the primers initiate the copying of the selected DNA using nucleotides and, typically, Taq polymerase or the like. By cycling the temperature, the selected DNA is repetitively denatured and copied. A single copy of the selected DNA, even if mixed in with other, random DNA, can be amplified to obtain billions of replicates. The polymerase chain reaction can be used to detect and measure very small amounts of DNA and to create customized pieces of DNA. In some instances, linear amplification methods may be used as an alternative to PCR.

The term “polymorphism” as used herein refers to any genetic changes or sequence variants in a locus, including but not limited to single nucleotide polymorphisms (SNPs), methylation differences, short tandem repeats (STRs), single gene polymorphisms, point mutations, trinucleotide repeats, indels and the like.

Generally, a “primer” is an oligonucleotide used to, e.g., prime DNA extension, ligation and/or synthesis, such as in the synthesis step of the polymerase chain reaction or in the primer extension techniques used in certain sequencing reactions. A primer may also be used in hybridization techniques as a means to provide complementarity of a locus to a capture oligonucleotide for detection of a specific locus.

The term “research tool” as used herein refers to any composition or assay of the invention used for scientific enquiry, academic or commercial in nature, including the development of pharmaceutical and/or biological therapeutics. The research tools of the invention are not intended to be therapeutic or to be subject to regulatory approval; rather, the research tools of the invention are intended to facilitate research and aid in such development activities, including any activities performed with the intention to produce information to support a regulatory submission.

The terms “sequencing”, “sequence determination” and the like as used herein refers generally to any and all biochemical methods that may be used to determine the order of nucleotide bases in a nucleic acid.

The term “source contribution” as used herein refers to the relative contribution of two or more sources of nucleic acids within an individual. The contribution from a source is generally determined as a percent of the nucleic acids from a sample, although any relative measurement can be used.

### DETAILED DESCRIPTION OF THE INVENTION

The methods described herein may employ, unless otherwise indicated, conventional techniques and descriptions of molecular biology (including recombinant techniques), cell biology, biochemistry, microarray and sequencing technology, which are within the skill of those who practice in the art. Such conventional techniques include polymer array synthesis, hybridization and ligation of oligonucleotides, sequencing of oligonucleotides, and detection of hybridization using a label. Specific illustrations of suitable techniques can be had by reference to the examples herein. However, equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, et al., Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Stryer, L., Biochemistry (4th Ed.) W.H. Freeman, New York (1995); Gait, “Oligonucleotide Synthesis: A Practical Approach” IRL Press, London (1984); Nelson and Cox, Lehninger, Principles of Biochemistry, 3^{rd} Ed., W. H. Freeman Pub., New York (2000); and Berg et al., Biochemistry, 5^{th} Ed., W.H. Freeman Pub., New York (2002), all of which are herein incorporated by reference in their entirety for all purposes. Before the present compositions, research tools and methods are described, it is to be understood that this invention is not limited to the specific methods, compositions, targets and uses described, as such may, of course, vary. It is also to be understood that the terminology used herein is for the purpose of describing particular aspects only and is not intended to limit the scope of the present invention, which will be limited only by appended claims.

It should be noted that as used herein and in the appended claims, the singular forms “a,” “an,” and “the” include plural referents unless the context clearly dictates otherwise. Thus, for example, reference to “a locus” refers to one, more than one, or mixtures of such regions, and reference to “an assay” includes reference to equivalent steps and methods known to those skilled in the art, and so forth.

Where a range of values is provided, it is to be understood that each intervening value between the upper and lower limit of that range—and any other stated or intervening value in that stated range—is encompassed within the invention. Where the stated range includes upper and lower limits, ranges excluding either of those included limits are also included in the invention.

Unless expressly stated, the terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated. All publications mentioned herein are incorporated by reference for the purpose of describing and disclosing the formulations and methodologies that are described in the publication and which might be used in connection with the presently described invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

### INVENTION IN GENERAL

The present invention provides methods for determining the fraction of fetal DNA in a maternal sample using massively parallel shotgun sequencing techniques. The invention utilizes a novel method of identifying informative polymorphisms identified through the sequencing process that align to designated regions in the genome. The fetal fraction can be determined by identifying a statistically significant number of these polymorphisms in multiple regions across the genome. The present invention also provides embodiments in which the fraction of fetal DNA in the maternal sample is determined by comparison of an observed distribution of all or a selected set of identified SNPs in a maternal sample to a fetal proportion reference comprised of distributions of these SNPs. When comparing an observed distribution of SNPs for a maternal sample to the fetal proportion reference, the distribution that most closely matches the observed distribution provides an estimate of the fetal fraction in the maternal sample.

In a preferred aspect, the polymorphisms used are single nucleotide polymorphisms (“SNPs”), and more preferably the SNPs are biallelic across populations, i.e., only two possible bases are observed at the SNP site in a polymorphic locus across the general populations. In certain aspects, the SNPs used are selected to be biallelic for a particular population (e.g., a geographic population) from which the maternal sample is obtained. While polymorphisms for use in the invention are described primarily in the specification with relation to the use of SNPs, it should be noted that other types of polymorphisms may be used in the present invention such as short tandem repeats (STRs), trinucleotide repeats, indels and the like.

Determination of the fraction of fetal DNA in a maternal sample has many beneficial uses in assessment of maternal and fetal condition. Depending on the embodiment, the value of the fraction of fetal DNA in a maternal sample may be useful in the determination of the presence of absence of fetal aneuploidy, as it provides important information on the expected statistical presence of nucleic acid regions and variation from that expectation may be indicative of copy number variation associated with insertions, deletions or aneuploidy. This may be particularly useful in circumstances where the level of fetal DNA in a maternal sample is low, as the fraction of fetal DNA in the sample can be used in determining the quantitative statistical significance in the variations of levels of identified nucleic acid regions. In other aspects, the determination of the fraction of fetal DNA in a maternal sample may be beneficial in estimating the level of certainty or power in detecting a fetal aneuploidy. Inaccurate estimation of fetal fraction of cell-free DNA contribution can lead to inaccurate determination of the presence or absence of fetal aneuploidy, leading to a false positive or a false negative result.

In certain aspects, determination of the fraction of fetal DNA in a maternal sample may be used to determine the number of fragments that should be randomly sequenced and/or the number of sequences that are to be analyzed based on a desired level of accuracy in a fetal aneuploidy determination. Fetal fraction in a maternal sample may alternatively, or in combination, be used as quality metric in which analyses of samples are only deemed acceptable when the fetal fraction is above a particular threshold. Alternatively, or in combination with any of the above, the fraction of fetal DNA in a maternal sample may itself be indicative of a disorder. For example, an unusually high fraction of fetal DNA in a maternal sample may be indicative of a physiological condition that causes an increase in DNA release from fetal and/or placental cells.

The methods of the present invention generally include conducting massively parallel DNA sequencing of random DNA fragments from a maternal sample which are then aligned to a reference to identify nucleic acids corresponding to single nucleotide polymorphisms (SNPs). The reference used can be, e.g., a consensus human genome sequence. The genomic reference is preferably a consensus sequence compiled from multiple individuals. In certain aspects, the reference may be a reference genomic sequence obtained from individuals in a population relevant to a particular maternal sample, e.g., a genomic reference sequence compiled from individuals of a particular race or geographic region. The reference can also be a database containing relevant SNP sequences, e.g., a database of biallelic SNPs. The reference may also be a collection of the haplotype information for tag SNPs that allow the haplotype to be imputed based on the identification of a particular tag SNP. The relative frequency of the SNPs are determined and used to calculate the fraction of fetal DNA in the maternal sample.

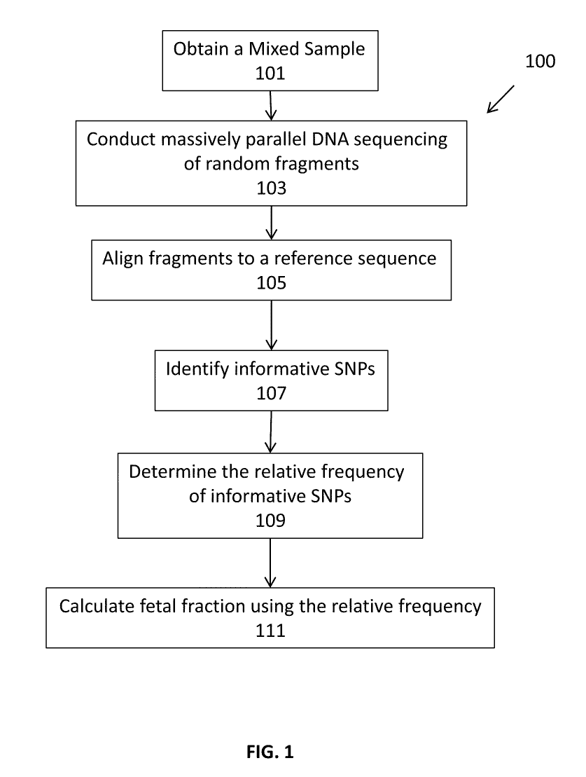

FIG. 1 is a simplified flow chart of the general steps utilized in determination of fetal fraction of cell-free DNA in a maternal sample in accordance with certain embodiments. FIG. 1 shows method 100, where in a first step 101 a maternal sample is obtained from a pregnant woman comprising maternal and fetal cell-free DNA. The maternal sample may be in any suitable form such as whole blood, plasma, serum, amniotic fluid, and tissue. In preferred embodiments, the sample comprises maternal plasma or serum. Depending on the type of sample used, additional processing and/or purification steps may be performed to obtain nucleic acid fragments of a desired purity or size, using processing methods including but not limited to sonication, nebulization, gel purification, PCR purification systems, nuclease cleavage, or a combination of these methods. Optionally, the cell-free DNA is isolated from the sample prior to further analysis.

At step 103, massively parallel DNA sequencing of random DNA fragments is conducted on the maternal sample to determine the sequence of the DNA fragments. At step 105, the fragment sequences are aligned to a reference. At step 107, nucleic acids corresponding to a plurality of SNPs are identified. In certain embodiments, steps 105 and 107 are performed simultaneously. In step 109, the relative frequency of the SNPs are determined. In step 111, the fetal fraction of the maternal sample is calculated using the relative frequency of the SNPs.

In certain embodiments, the methods of the present invention also include determination of the presence or absence of fetal aneuploidy. These methods include conducting massively parallel DNA sequencing of random DNA fragments from a maternal sample which are then aligned to a reference to identify nucleic acids corresponding to a first chromosome and a second chromosome, preferably a chromosome of interest and a reference chromosome. The relative frequency of the DNA fragment sequences of a chromosome of interest are compared to the relative frequency of DNA fragment sequences from a reference to determine the presence or absence of fetal aneuploidy by detecting a copy number variation in all or a portion of the chromosome of interest. The fetal aneuploidy can be any full or partial aneuploidy such as a trisomy, monosomy, mosaicism, translocations, deletions, insertions, etc. In certain preferred embodiments, the chromosome tested for being aneuploidy is chromosome 13, chromosome 18, chromosome 21, chromosome X or chromosome Y.

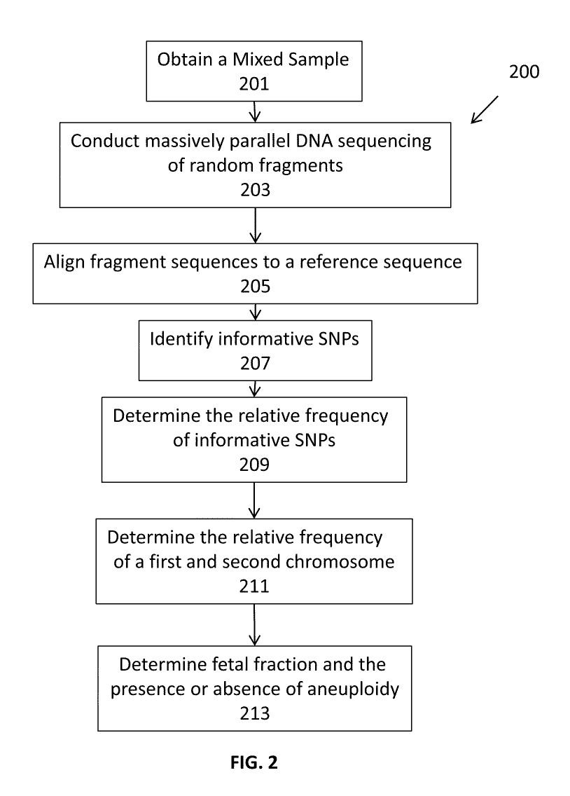

Depending on the embodiment, determination of fetal fraction of the maternal sample and determination of the presence or absence of fetal aneuploidy may be performed simultaneously. FIG. 2 is a simplified flow chart of the general steps utilized in the simultaneous determination of the presence or absence of fetal aneuploidy and fetal fraction in a maternal sample. FIG. 2 shows method 200 where in a first step 201 a maternal sample is obtained from a pregnant woman comprising maternal and fetal cell-free DNA. The maternal sample may be in any suitable form such as whole blood, plasma, serum, amniotic fluid, and tissue. In preferred embodiments, the sample comprises maternal plasma or serum. Depending on the type of sample used, additional processing and/or purification steps may be performed to obtain nucleic acid fragments of a desired purity or size, using processing methods including but not limited to sonication, nebulization, gel purification, PCR purification systems, nuclease cleavage, or a combination of these methods. Optionally, the cell-free DNA is isolated from the sample prior to further analysis.

At step 203, massively parallel DNA sequencing of random DNA fragments is conducted on the maternal sample to determine the sequence of the DNA fragments. At step 205, the fragment sequences are aligned to a reference. At step 207, nucleic acids corresponding to a plurality of SNPs are identified. In certain embodiments, steps 205 and 207 are performed simultaneously. In step 209, the relative frequency of SNPs is determined. In a step not shown, nucleic acids corresponding to a first chromosome and nucleic acids corresponding to a second chromosome are identified. This step may be performed simultaneously with step 207, before step 207 or after. In step 211, the relative frequency of a first chromosome and a second chromosome are determined. In step 213, the fetal fraction and the presence or absence of fetal aneuploidy are determined. Optionally, in certain embodiments, the fetal fraction and the presence or absence of fetal aneuploidy may be determined sequentially.

Determination of the presence or absence of fetal aneuploidy may comprise comparing the relative frequency of a first chromosome to the relative frequency of a second chromosome. In certain embodiments, a first chromosome may be a chromosome of interest suspected of being aneuploid while the second chromosome is a reference chromosome that is not suspected of being aneuploid. These concepts will be discussed in further detail below.

In certain embodiments, a likelihood of a fetal chromosomal abnormality is statistically determined. Statistically determining the likelihood of a fetal chromosomal abnormality may comprise comparing the relative frequency of a first chromosome to the relative frequency of a second chromosome. In certain embodiments, the likelihood calculation is based on a likelihood that a fetal genomic region is disomic and a likelihood that the fetal genomic region is not disomic, such as a likelihood that the fetal genomic region is trisomic or monosomic. The likelihood of a fetal chromosomal abnormality may be adjusted or calculated using the fetal fraction of the maternal sample. Such methods are described, e.g., in U.S. Ser. No. 13/316,154, filed 9 Dec., 2011; U.S. Ser. No. 13/338,963, filed 28 Dec. 2011; U.S. Ser. No. 13/356,133, filed 23 Jan. 2012; U.S. Ser. No. 13/356,575, filed 23 Jan. 2012; U.S. Ser. No. 13/553,012, filed 19 Jul. 2012; U.S. Ser. No. 13/605,505, filed 6 Sep. 2012; U.S. Ser. No. 13/689,206, filed 29 Nov. 2012; and U.S. Ser. No. 13/689,417, filed 29 Nov. 2012.

### SEQUENCING METHODS

In the present invention, massively parallel shotgun sequencing is used to sequence random fragments of both fetal and maternal DNA of a mixed maternal sample. Massively parallel sequencing of random DNA fragments allows sequencing of large portions of the fetal genome, which can be particularly useful in the sequencing of maternal samples as the fetal DNA is generally present in low concentrations in comparison to the maternal DNA. Sequencing of large portions of the genome can increase the sensitivity and specificity of the sequencing to achieve a desired level of accuracy of subsequent analyses as it can increase the amount of information from the fetal sequences that are available in low abundance in comparison to other techniques. The number of random DNA fragments that are sequenced may be determined or adjusted in view of the fetal fraction in the maternal sample. This will be described in greater detail below.

Massively parallel shotgun sequencing may be performed using any suitable sequencing apparatus capable of sequencing many fragments from samples at high orders of multiplexing such as the miSeq (Illumina), Ion PGM™ (Life Technologies), HiSeq 2000 (Illumina), HiSeq 2500 (Illumina), 454 platform (Roche), Illumina Genome Analyzer (Illumina), SOLiD System (applied Biosystems), Helicos True Single Molecule DNA sequencer (Helicos), real-time SMRT™ technology (Pacific Biosciences) and suitable nanopore sequencers.

Massively parallel sequencing of random DNA fragments provides fragment sequences that reflect the profile of the original sample. Sequencing is performed such that statistically less than the full genome is sequenced. Depending on the level of sequencing performed, statistically, each section of nucleic acids is sequenced multiple times. The higher the level of sequencing performed, the higher the resulting level of redundancy in the sampling of nucleic acid regions of the genome which provides a more accurate reflection of the frequency of nucleic acid sequences in the original sample.

In certain embodiments, all of the fragments from a maternal sample are sequenced, while in other embodiments only a subset of the fragments of a sample are sequenced. The subset of fragments may be chosen at random or the subset may be chosen based on specific parameters to maximize accuracy of analysis. For example, in certain embodiments, only a subset of fragments that are of a particular size are sequenced. Filtering of fragments based on size may be carried out using any suitable method such as hybridization techniques, gel electrophoresis, size exclusion columns, or microfluidics. In other certain embodiments, a subset of fragment sequences may be selected from the sequencing results to be aligned to reference and carried through subsequent steps of the analysis.

In certain embodiments, portions of the sample may be enriched prior to sequencing. For example, fetal fragments may be enriched prior to sequencing to reduce the number of overall fragments that need to be analyzed to obtain a desired level of accuracy.

In random sequencing, the number of sequences to be obtained may be determined prior to performing the sequencing operation. For example, a number of sequences to be performed on a sample may be determined based on the fraction of fetal DNA in the sample. The number of sequence reads performed may be increased if the fraction of fetal DNA in the maternal sample is small. Conversely, the number of sequence reads performed in the sequencing operation may be decreased if there is a higher abundance of fetal DNA in the maternal sample. In other embodiments, the number of sequence reads may be determined independently without regard for the fraction of fetal DNA in the maternal sample.

As will be described in greater detail below, the number of fragment sequences used to determine the fraction of fetal DNA in the sample may be determined by the amount of data required to obtain a statistically significant estimation of fetal fraction. In certain embodiments, less than 100% of the genome may be sequenced, such as less than 50% of the genome, or less than 20% of the genome. In certain aspects, massively parallel sequencing of random DNA fragments produces between one million and ten million fragment sequences. In certain embodiments, the sequence obtained from the random DNA fragments is from about 15 bp to about 150 bp in length, more preferably from about 25 bp to about 100 bp in length.

In certain embodiments, only one end of each fragment is sequenced while in other certain embodiments both ends of each fragment are sequenced. In other embodiments, each entire fragment is sequenced. In further certain embodiments, sequencing may be performed using paired end sequencing.

Samples may be multiplexed in the sequencing process. For example, in certain embodiments, five or more samples may be pooled in a single sequencing process, or more preferably ten or more samples, or more preferably twenty or more samples, or more preferably fifty or more samples or even more preferably ninety or more samples.

Once fragment sequences are obtained, they are identified as corresponding to specific locations of the genome, for example by aligning the sequenced DNA fragments to a reference.

Any suitable technique may be used to correct for variance in levels found between samples and/or for informative loci within a sample caused by factors such bias in the sequencing process. For example, an internal reference, such as a chromosome present in a “normal” abundance (e.g., disomy for an autosome) to compare against a chromosome present in a putatively abnormal abundance, such as aneuploidy in the sample. While the use of one such “normal” chromosome as a reference chromosome may be sufficient, it is also possible to use two to many normal chromosomes as the internal reference chromosomes to increase the statistical power of the quantification.

### Calculating Fetal Fraction Using Relative Frequencies of SNPs

Calculation of the fraction of fetal DNA in the maternal sample comprises identification and quantification of polymorphisms in the maternal and fetal genome, such as SNPs. The SNPs are identified using information collected in the sequencing and alignment processes described above. The fetal fraction can be calculated by determining the relative frequency of the SNPs, using a statistically significant number of SNPs in multiple designated regions across the genome.

In certain embodiments, the percent fetal DNA in the maternal sample is determined in multiple designated regions comprising SNPs to increase the accuracy of the calculation, rather than using a single region of SNPs to represent the entire genome. The number and size of the designated regions may vary depending on the embodiment and the chromosome being evaluated. For example, the higher the concentration SNPs contained in a particular area of the genome, the smaller the size of the designated regions required for accurate calculation of the fetal fraction of DNA in the sample. Conversely, the lower the concentration of SNPs contained in a particular area of the genome, the larger the designated regions required for accurate calculation of the fraction of fetal DNA in the sample. Each designated region should be of sufficient size to contain a requisite number of SNPs for the calculation of the fetal fraction to be statistically significant. The accuracy of the calculation of fetal fraction is dependent upon the number of SNPs in each designated region and thus, the present invention preferably further comprises determining the number of SNPs required to determine fetal fraction in maternal samples.

The number of SNPs required for statistically significant calculation of fetal fraction also depends on the level of multiplexing of samples in the sequencing process. For example, the number of SNPs required to determine fetal fraction in samples multiplexed on hundred-fold in the sequencing process is on the order of 10 times greater than the number of SNPs required to determine fetal fraction in samples multiplexed fifty-fold in the sequencing process.

Accounting for both the level of multiplexing and the desired level of accuracy of the calculation of fraction fetal, in certain embodiments the number of SNPs required to achieve a statistically significant estimation of the fraction of fetal DNA in a maternal sample is determined by comparison to a fetal proportion reference comprised of SNP information. The number of SNPs required to accurately calculate the fraction of fetal DNA in a maternal sample may vary widely depending on the particular sample.

The size of the designated regions may vary widely in each analysis due to variance in the distribution of SNPs throughout the genome.

In certain embodiments, SNPs used in the present invention include any SNP identified through random sequencing detection processeses. In other certain embodiments, SNPs used in the analysis are informative SNPS. In certain aspects, informative SNPs include any SNP where the maternal allele differs from the fetal allele. In other certain aspects, informative SNPs include any SNP in which the maternal allele is homozygous and the fetal allele is heterozygous.

In certain embodiments, the informative SNPs are tag SNPs. A “tag SNP” is a representative single nucleotide polymorphism (SNP) in a region of the genome with high linkage disequilibrium, i.e. the non-random association of alleles at two or more loci. Alleles of SNPs in close physical proximity to each other are often correlated, and the variation of the sequence of alleles in contiguous SNP sites along a chromosomal region is known to be of limited diversity. It is thus possible to determine multiple SNPs associated with a tag SNP without genotyping every SNP in the nucleic acid region. Tag SNPs are particularly useful in whole-genome SNP association studies in which hundreds of thousands of SNPs across the entire genome are genotyped, as they provide information about multiple SNPs in a nucleic acid region.

Tag SNPs can be identified using methods known to those skilled in the art. For example, algorithms are available that predict the values of the SNPs of a haplotype upon identification of a single tag SNPs. See, e.g., IdSelect (Carlson et al., Am. J. Human Genet., 2004, 74, 106-120) and HapBlock (Zhang et al., Genome Res., 2004 14, 908-916.). In another example, an algorithm can be used which utilizes the genotype values of the tag SNPs, such as STAMPA. See, e.g., Halperin E et al., Bioinformatics. 2005 June; 21 Suppl 1:i195-203.

Because of their association with other SNPs in a haplotype, using tag SNPs requires fewer SNPs used in determining the fetal fraction to achieve a statistical significant result. Because a single tag SNP is indicative of one or more associated SNP sites, fewer tag SNPs are necessary to achieve a statistically significant number of SNPs for the determination of fetal fraction in a maternal sample. For example, if in a multiplexed sample set of 10, it would require 100 single SNPs per designated region to calculate a statistically significant determination of the fetal fraction of each sample, while using tag SNPs that are indicative of 4 individual SNPs (including the tag SNP) only 25 such tag SNPs would be required to reach the same statistical significance. The use of tag SNPs may also decrease the size of the designated regions used in the calculation of fetal fraction.

The use of tag SNPs also allows a greater level of multiplexing of samples compared to non-tag SNPs while using the same number of SNPs in the evaluation. For example, if in a multiplexed sample set of 10, it would require 100 single SNPs per designated region to calculate a statistically significant determination of the fetal fraction of each sample, while using 100 tag SNPs per designated region would allow the multiplexing of 40 samples with the same statistical significance.

The fraction of fetal DNA in the maternal sample is determined in certain embodiments by comparison of an observed distribution of SNPs in a maternal sample to a fetal proportion reference. The fetal proportion reference is a set of expected SNP distributions at various fetal fraction levels. When comparing an observed distribution of SNPs for a maternal sample to the fetal proportion reference, the distribution that most closely matches the observed distribution provides an estimate of the fetal fraction in the maternal sample.

The fetal proportion reference that is used in the comparison may be generated using empirical or simulated information. Simulated distributions for different fetal fractions can be used to create a fetal proportion reference, e.g., based on mathematical modeling or graphical modeling for different fetal fractions. In certain embodiments, a fetal proportion reference is based on the expected level of SNPs distributions in the population and the expected number of fragments analyzed from a given MPSS procedure to analyze maternal and fetal genomic DNA. These simulated distributions can be directly compared to the empirical data obtained from an MPSS analysis of the cell-free DNA of a maternal sample, and the fetal fraction for a maternal sample estimated based on concordance with a simulated distribution for the SNPs in the fetal proportion reference.

Alternatively, a compilation of observed distributions from multiple maternal samples of known fetal fraction may be used to create a fetal proportion reference. These compilations would comprise data from maternal samples analyzed for the fetal fraction to obtain a consensus distribution at various fetal fractions. An observed distribution for SNPs analyzed by MPSS performed on a maternal sample is compared to a fetal proportion reference of consensus distributions, and the distribution most closely matching the observed distributions of the maternal sample would be used to estimate the fetal fraction in that maternal sample.

Empirical data from a particular sample are compared to the fetal proportion reference to estimate the fetal fraction of that particular sample. The obtained reads for the SNPs of an individual sample with greater than 8 counts is compared with models of the distributions generated through simulations with different fetal fractions. These comparisons are made using a variety of techniques, e.g., comparing simulated data and parameter estimation techniques including expectation maximization. The fetal fraction parameter for the model that best matches the observed distribution of fractions as set forth in the fetal proportion reference provides an estimate of the fetal fraction in the individual samples.

Depending on the embodiment, it is not necessary for all sequences to be used in the calculation of fetal fraction. For example, only those sequences that are aligned specific nucleic acids regions, such as specific designated regions, may be used in the calculation of fetal fraction. Alternatively, or in combination, only those sequences that fall within certain quality parameters may be used in further analysis. For example, a subset of sequences of a certain size may be selected for further analysis. A subset of sequences may also be selected based on their location on particular chromosomes.

There are many other standard methods for choosing the subset of sequences. These methods include outlier exclusion, where the fragments with detected levels below and/or above a certain percentile are discarded from the analysis. In one aspect, the percentile may be the lowest and the highest 5% as measured by abundance. In another aspect, the percentile may be the lowest and highest 10% as measured by abundance. In another aspect, the percentile may be the lowest and highest 25%.

Another method for choosing the subset of sequences includes the elimination of regions that fall outside of some statistical limit. For instance, sequences that fall outside of one or more standard deviations of the mean abundance may be removed from the analysis. Another method for choosing the subset of sequences may be to compare the relative abundance of sequences to the expected abundance of the same sequence in a healthy population and discard any sequences that fail the expectation test.

In another aspect, subsets of sequences can be chosen randomly but with sufficient numbers of sequences to yield a statistically significant result in determining whether a chromosomal abnormality exists. Multiple analyses of different subsets of sequences can be performed within a mixed sample to yield more statistical power. In this example, it may or may not be necessary to remove or eliminate any sequences prior to the random analysis. For example, if there are 100 fragment sequences for chromosome 21 and 100 fragment sequences for chromosome 18, a series of analyses could be performed that evaluate fewer than 100 sequences for each of the chromosomes.

### Determining the Presence or Absence of an Aneuploidy

The present invention further comprises a method for the determination of the presence or absence of fetal aneuploidy. In certain preferred embodiments, the determination of the presence or absence of fetal aneuploidy may be performed simultaneously with the determination of the fraction of fetal DNA in a sample. In other embodiments, these determinations may be performed sequentially.

Based on the information obtained from aligning the fragment sequences to a reference, fragment sequences are identified as corresponding to nucleic acid regions on specific chromosomes in the maternal and fetal DNA. The relative frequency of fragment sequences identified as corresponding to a first chromosome, preferably a chromosome of interest, is compared to the relative frequency of fragment sequences identified as corresponding to a second chromosome, preferably a reference chromosome. Aneuploidy can then be determined by detecting an over-representation of the chromosome of interest compared to the reference chromosome.

One example of calculating a relative frequency comprises determining the abundance or counts of fragment sequences (or selected subset of fragment sequences) for each chromosome or a portion of a chromosome which are summed together to calculate the total counts for each chromosome and then comparing the sum for one chromosome to the total sum for another chromosome.

Alternatively, a relative frequency for each chromosome may be calculated by first summing the counts of the fragment sequences or selected subset of fragment sequences for each chromosome and then comparing the sum for one chromosome to the total sum for two or more chromosomes. Once calculated, the relative frequency is then compared to the average relative frequency from a normal population.

The average may be the mean, median, mode or other average, with or without normalization and exclusion of outlier data. In a preferred aspect, the mean is used. In developing the data set for the relative frequency from the normal population, the normal variation of the measured chromosomes is calculated. This variation may be expressed a number of ways, most typically as the coefficient of variation, CV. When the relative frequency from the sample is compared to the average relative frequency from a normal population, if the relative frequency for the sample falls statistically outside of the average relative frequency for the normal population, the sample contains an aneuploidy.

In certain embodiments, a relative frequency may be determined by calculating the average counts of fragment sequences for each chromosome. The average may be any estimate of the mean, median or mode, although typically an average is used. The average may be the mean of all counts or some variation such as a trimmed or weighted average. Once the average counts for each chromosome have been calculated, the average counts for each chromosome may be compared to another to obtain a chromosomal ratio between two chromosomes, the average counts or each chromosome may be compared to the sum of the averages for more than two chromosomes, such as all measured chromosomes to obtain a relative frequency for each chromosome as described above.

The ability to detect an aneuploidy in a maternal sample where the putative DNA is in low relative abundance depends greatly on the variation in the measurements of different chromosomes. Numerous analytical methods can be used which reduce this variation and thus improve the sensitivity of this method to detect aneuploidy. One method for reducing variability of the assay is to increase the number of fragment sequences used to calculate the abundance of chromosomes.

In one aspect, following the measurement of abundance for the fragments of each chromosome, a subset of sequences may be selected and used in the determination of the presence of absence of fetal aneuploidy. There are many standard methods for choosing the subset of sequences. These methods include outlier exclusion, where the fragments with detected levels below and/or above a certain percentile are discarded from the analysis. In one aspect, the percentile may be the lowest and the highest 5% as measured by abundance. In another aspect, the percentile may be the lowest and highest 10% as measured by abundance. In another aspect, the percentile may be the lowest and highest 25%.

Another method for choosing the subset of sequences includes the elimination of regions that fall outside of some statistical limit. For instance, sequences that fall outside of one or more standard deviations of the mean abundance may be removed from the analysis. Another method for choosing the subset of sequences may be to compare the relative abundance of sequences to the expected abundance of the same sequence in a healthy population and discard any sequences that fail the expectation test.

In another aspect, subsets of sequences can be chosen randomly but with sufficient numbers of sequences to yield a statistically significant result in determining whether a chromosomal abnormality exists. Multiple analyses of different subsets of sequences can be performed within a mixed sample to yield more statistical power. In this example, it may or may not be necessary to remove or eliminate any sequences prior to the random analysis. For example, if there are 100 fragments for chromosome 21 and 100 sequences for chromosome 18, a series of analyses could be performed that evaluate fewer than 100 sequences for each of the chromosomes.

In another aspect, subsets can be chosen by their location on a particular chromosome. For example, only those sequences that are aligned to a first chromosome of interest and a reference chromosome may be used in the determination. Alternatively, only those sequences that are aligned to a first chromosome of interest and those sequences that are aligned to a predetermined number of reference chromosomes may be used for determination fetal aneuploidy. Alternatively, or in combination, only those sequences that fall within certain quality parameters may be used in further analysis. For example, a subset of sequences of a certain size may be selected for further analysis.

In certain embodiments, determination of the presence or absence of fetal aneuploidy may be performed in view of a cutoff value. For example, the difference in relative frequencies between the first chromosome and the second chromosome may be compared to a cutoff value to determine if the difference is large enough to signify the presence of a fetal aneuploidy. In other embodiments, a risk score for the presence or absence of fetal aneuploidy may be calculated for each sample using the relative frequencies of the first and second chromosomes. In these embodiments, the calculated fraction of fetal DNA in the sample may be used in the calculation of a risk score for the presence or absence of fetal aneuploidy.

The criteria for setting the cutoff value to declare an aneuploidy depend on the variation in the measurement of the relative frequency and the acceptable false positive and false negative rates for the methods. In general, this cutoff may be a multiple of the variation observed in the relative frequency.

In certain embodiments, a likelihood of a fetal chromosomal abnormality is statistically determined. Statistically determining the likelihood of a fetal chromosomal abnormality may comprise comparing the relative frequency of a first chromosome to the relative frequency of a second chromosome. In certain embodiments, the likelihood calculation is based on a likelihood that a fetal genomic region is disomic and a likelihood that the fetal genomic region is not disomic, such as a likelihood that the fetal genomic region is trisomic or monosomic. The likelihood of a fetal chromosomal abnormality may be adjusted or calculated using the fetal fraction of the maternal sample.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1

### Sample Procurement

Subjects were prospectively enrolled upon providing informed consent, under protocols approved by institutional review boards. Subjects were required to be at least 18 years of age, at least 10 weeks gestational age, and to have singleton pregnancies. A subset of enrolled subjects, consisting of 250 women was selected for inclusion in this study. The subjects were randomized until after analysis.

8 mL blood per subject was collected into a Cell-free DNA tube (Streck, Omaha, Nebr.) and stored at room temperature for up to 3 days. Plasma was isolated from blood via double centrifugation and stored at −20° C. for up to a year. cfDNA was isolated from plasma using Viral NA DNA purification beads (Life Technologies, Carlsbad, Calif.), biotinylated, immobilized on MyOne C1 streptavidin beads (Life Technologies, Carlsbad, Calif.). The DNA from each sample was prepared for sequencing using a TruSeq™ DNA PCR-Free HT Sample Preparation Kit (Illumina, San Diego Calif.) for high-throughput studies. This preparation provides library preparation for each sample, including 96 dual indices that allow identification of the individual samples within the sequencing run.

### Example 2

### Determination of Fetal Fraction in a Maternal Sample Using MPSS

Massively parallel shotgun sequencing (MPSS) of the prepared DNA obtained as per Example 1 is performed using an Illumina HiSeg™ instrument and the associated reagents. Briefly, the prepared DNA of each sample is run on a single HiSeq lane. 160,000,000 mapped reads are obtained from the sequencing run, each approximately 36 nucleotides (nts) in length. As of dbSNP Build 137, there are more than 50,000,000 reference SNPs in the human genome. Assuming a Poisson distribution of reads across the human genome with a mean of 160,000,000*36/3,000,000,000 reads mapping to a genomic position (which has been observed by Fan and Quake, 2010), 40,000 reference SNPs are identified each having at least 8 reads. Although each individual SNP has a small number of reads, having 40,000 or more observations provides enough statistical power to detect distributional differences leading to estimates for fetal fraction.

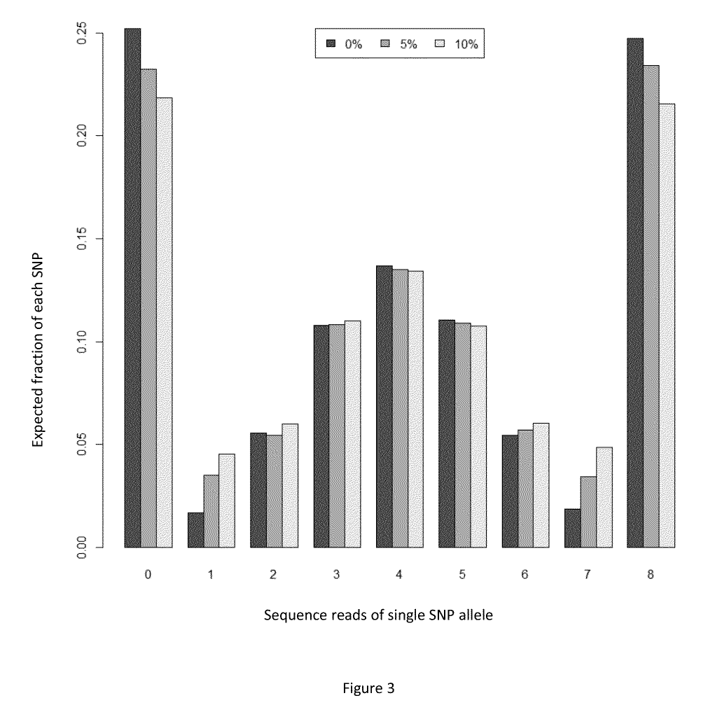

For each SNP, the distribution of fractions can be determined by a/(a+b), where a represents the number of counts for the less abundant allele (e.g. A for an A/C variant, C for a C/G variant, etc.) and b represents the number of counts for the more abundant allele. Simulated distributions for different fetal fractions can be used to create a fetal proportion reference, e.g., based on mathematical modeling or graphical modeling for different fetal fractions. An exemplary fetal proportion reference is depicted in FIG. 3, which illustrates graphical distributions based on simulated distributions from calculations using 40,000 reference SNPs. This graphical illustration of a fetal proportion reference is based on the expected level of SNPs distributions in the population and the expected number of fragments analyzed from a given MPSS procedure to analyze maternal and fetal genomic DNA. The X axis of FIG. 3 represents the expected sequence reads that would be obtained for one of two possible alleles for a SNP at a biallelic locus resulting from MPSS analysis of cell-free DNA obtained from a maternal sample. The Y axis represents the fraction of fragments analyzed expected to contain a SNP from each biallelic locus. These simulated distributions can be directly compared to the empirical data obtained from an MPSS analysis of the cell-free DNA of a maternal sample, and the fetal fraction for a maternal sample estimated based on concordance with a simulated distribution for the SNPs in the fetal proportion reference.

Alternatively, a compilation of observed distributions from multiple maternal samples of known fetal fraction may be used to create a fetal proportion reference. These compilations would comprise data from maternal samples analyzed for the fetal fraction to obtain a consensus distribution at various fetal fractions. An observed distribution for SNPs analyzed by MPSS performed on a maternal sample is compared to a fetal proportion reference of consensus distributions, and the distribution most closely matching the observed distributions of the maternal sample would be used to estimate the fetal fraction in that maternal sample.

Empirical data from a particular sample are compared to the fetal proportion reference to estimate the fetal fraction of that particular sample. The obtained reads for the SNPs of an individual sample with greater than 8 counts is compared with models of the distributions generated through simulations with different fetal fractions. These comparisons are made using a variety of techniques, e.g., comparing simulated data and parameter estimation techniques including expectation maximization. The fetal fraction parameter for the model that best matches the observed distribution of fractions as set forth in the fetal proportion reference provides an estimate of the fetal fraction in the individual samples.

### Example 3

### Determination of Fetal Fraction in a Maternal Sample Using MPSS and Informative SNPs

MPSS of the prepared DNA obtained as per Example 1 is performed using an Illumina MiSeg™ instruments and the associated reagents. Briefly, the prepared DNA of each sample is prepared on a single MiSeq lane. Approximately 18,000,000 mapped reads are obtained from the sequencing run, each approximately 36 nucleotides (nts) in length. Assuming a Poisson distribution of reads across the human genome (which has been observed by Fan and Quake, 2010), fewer than 1 SNP would be expected to have even 6 mapped reads for any given MPSS run.

To overcome this lack of depth and corresponding lack of statistical power, reads for SNPs can be aggregated together when they are known to be in high linkage disequilibrium, where observing the reads for one SNP are highly predictive of a corresponding read on another SNP. Information regarding SNPs in high linkage disequilibrium are available from the HAPMAP and 1000 Genomes projects.

As a rough example, suppose that SNP groups are created out of 100 reference SNPs, giving rise to 500,000 SNP groups. In this setting, we expect a mean number of reads mapping to a group of 100 positions to be roughly 18,000,000*3600/3,000,000,000, approximately 21 reads per group. Using a Poisson distribution, more than 499,000 SNP groups are expected to have at least 8 reads and more than 16,000 can be expected to have at least 30 reads. Using expectation maximization, it is possible once again to estimate the fetal fraction that would give rise to the allele distributions contained in the SNP groups.

Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. §112, ¶6.

What is claimed is:

1. A method for determining fetal fraction in a maternal sample, wherein the method comprises:
a. obtaining a mixture of fetal and maternal genomic DNA from said maternal sample;
b. conducting massively parallel DNA sequencing of random DNA fragments from the mixture of fetal and maternal genomic DNA of step a) to determine the sequence of said DNA fragments;
c. identifying nucleic acids corresponding to a plurality of informative single nucleotide polymorphisms in designated regions of the genomic DNA by alignment of the sequenced DNA fragments to a reference;
d. determining the relative frequency of the sequenced informative single nucleotide polymorphisms; and
e. calculating the fetal fraction of the maternal sample using the relative frequency of the sequenced informative single nucleotide polymorphisms.

2. The method of claim 1, wherein the informative single nucleotide polymorphisms are used to impute haplotype information to distinguish maternal and fetal DNA.

3. The method of claim 1, wherein the sequence of the DNA fragments is from about 15 bp to about 150 bp in length.

4. The method of claim 3, wherein the sequence of the DNA fragments is from about 25 bp to about 100 bp in length.

5. The method of claim 1, wherein the genomic DNA is cell-free DNA.

6. The method of claim 4, wherein the maternal sample is maternal plasma or serum.

7. The method of claim 1, further comprising determining the number of informative single nucleotide polymorphisms necessary for a statistically significant estimation of fetal fraction in the maternal sample.

8. A method for determination of fetal fraction in five or more maternal samples, comprising:
a. obtaining a mixture of random fragments of fetal and maternal genomic DNA from each maternal sample;
b. introducing sample indices unique to the individual samples to the random fragments of each sample;
c. conducting massively parallel DNA sequencing of random DNA fragments from the mixture of fetal and maternal genomic DNA of each maternal sample to determine the sequence of said DNA fragments;
d. identifying nucleic acids corresponding to a plurality of informative SNPs in designated regions of the genomic DNA by alignment of the sequenced DNA fragments of each sample to a reference;
e. identifying the number of informative SNPs necessary to obtain a statistically significant estimation of fetal fraction in each of the maternal samples;
f. determining the relative frequency of at least the identified number of sequenced informative SNPs in each sample, wherein the informative SNPs for an individual sample are identified using the sample index; and
g. calculating the fetal fraction of the individual maternal samples using the relative frequency of the sequenced informative single nucleotide polymorphisms.

9. The method of claim 8, wherein the method determines the fetal fraction of ten or more maternal samples.

10. The method of claim 9, wherein the method determines the fetal fraction of twenty or more maternal samples.

11. The method of claim 10, wherein the method determines the fetal fraction of fifty or more maternal samples.

12. The method of claim 11, wherein the method determines the fetal fraction of ninety or more maternal samples.

13. The method of claim 8, wherein the sequence of the DNA fragments is from about 15 bp to about 150 bp in length.

14. The method of claim 13, wherein the sequence of the DNA fragments is from about 25 bp to about 100 bp in length.

15. The method of claim 8, wherein the genomic DNA is cell-free DNA.

16. The method of claim 15, wherein the maternal sample is maternal plasma or serum.

17. The method of claim 8, wherein the informative single nucleotide polymorphisms are tag single nucleotide polymorphisms.

18. A method for determining fetal fraction in a maternal sample, wherein the method comprises:
a. obtaining a mixture of fetal and maternal genomic DNA from said maternal sample;
b. conducting massively parallel DNA sequencing of random DNA fragments from the mixture of fetal and maternal genomic DNA of step a) to determine the sequence of said DNA fragments;
c. identifying nucleic acids corresponding to a plurality of tag single nucleotide polymorphisms by alignment of the sequenced DNA fragments to a reference;
d. determining the relative frequency of the sequenced tag single nucleotide polymorphisms; and
e. calculating the fetal fraction of the maternal sample using the relative frequency of the sequenced tag single nucleotide polymorphisms.

19. The method of claim 18, wherein the reference to which the sequenced DNA fragments are aligned comprises one or more reference genomes.

20. The method of claim 18, wherein the reference to which the sequenced DNA fragments are aligned comprises a single nucleotide polymorphism database.

21. The method of claim 18, wherein the informative single nucleotide polymorphisms are used to impute haplotype information to distinguish maternal and fetal DNA.

22. The method of claim 18, wherein the sequence of the DNA fragments is from about 15 bp to about 150 bp in length.

23. The method of claim 22, wherein the sequence of the DNA fragments is from about 25 bp to about 100 bp in length.

24. The method of claim 18, wherein the genomic DNA is cell-free DNA.

25. The method of claim 24, wherein the maternal sample is maternal plasma or serum.

26. The method of claim 18, further comprising determining the number of tag SNPs necessary for a statistically significant estimation of fetal fraction in the maternal sample.

27. A method for simultaneously determining the presence or absence of a fetal aneuploidy and fetal fraction in a maternal sample, wherein the method comprises:
a. obtaining a mixture of fetal and maternal genomic DNA from a maternal sample;
b. conducting massively parallel DNA sequencing of random DNA fragments from the mixture of fetal and maternal genomic DNA of step a) to determine the sequence of said DNA fragments;
c. aligning the DNA fragment sequences generated from step b) to a reference;
d. determining a relative frequency of DNA fragment sequences corresponding to a plurality of informative single nucleotide polymorphisms based on the alignment of the DNA fragment sequences to the reference;
e. determining a relative frequency of DNA fragment sequences from a first chromosome based on the alignment of the DNA fragment sequences to the reference;
f. determining a relative frequency of DNA fragment sequences from a second chromosome based on the alignment of the DNA fragment sequences to the reference; and
g. determining the fetal fraction of the maternal sample using the relative frequency of the sequenced informative single nucleotide polymorphisms and the presence or absence of a fetal aneuploidy using the relative frequencies of DNA fragment sequences from the first and second chromosome.

28. The method of claim 27, wherein the fetal fraction is a quality control metric, and wherein the fetal aneuploidy is only determined if fetal fraction is above a cut-off.

29. The method of claim 27, wherein the fetal fraction is used in the calculation to determine the presence or absence of fetal aneuploidy.

30. The method of claim 27, wherein the sequence of the DNA fragments is from about 15 bp to about 150 bp in length.

31. The method of claim 30, wherein the sequence of the DNA fragments is from about 25 bp to about 100 bp in length.

32. The method of claim 27, wherein the genomic DNA is cell-free DNA.

33. The method of claim 32, wherein the maternal sample is maternal plasma or serum.

34. The method of claim 27, wherein the fetal aneuploidy is an aneuploidy selected from the group consisting of chromosome 13, chromosome 18, chromosome 21, chromosome X and chromosome Y.

35. The method of claim 27, wherein the informative single nucleotide polymorphisms are tag single nucleotide polymorphisms.

36. A method for statistically determining the likelihood of a fetal chromosomal abnormality in a maternal sample comprising fetal and maternal cell-free genomic DNA, the method comprising:
a. obtaining a mixture of fetal and maternal genomic DNA from a maternal sample;
b. conducting massively parallel DNA sequencing of random DNA fragments from the mixture of fetal and maternal genomic DNA of step a) to determine the sequence of said DNA fragments;
c. aligning the DNA fragment sequences generated from step b) to a reference;
d. determining a relative frequency of DNA fragment sequences corresponding to a plurality of informative single nucleotide polymorphisms based on the alignment of the DNA fragment sequences to the reference;
e. determining a relative frequency of DNA fragment sequences from a first chromosome based on the alignment of the DNA fragment sequences to the reference;
f. determining a relative frequency of DNA fragment sequences from a second chromosome based on the alignment of the DNA fragment sequences to the reference; and
g. determining the fetal fraction of the maternal sample using the relative frequency of the sequenced informative single nucleotide polymorphisms; and
h. statistically determining the likelihood of a fetal chromosomal abnormality based on the relative frequencies of DNA fragment sequences from the first and second chromosome.

37. The method of claim 36, wherein the fetal fraction is a quality metric, and wherein the fetal aneuploidy is only determined if fetal fraction is above a cut-off.

38. The method of claim 36, wherein the fetal fraction is used in the calculation to determine the presence or absence of fetal aneuploidy.

39. The method of claim 36, wherein the sequence of the DNA fragments is from about 15 bp to about 150 bp in length.

40. The method of claim 39, wherein the sequence of the DNA fragments is from about 25 bp to about 100 bp in length.

41. The method of claim 36, wherein the genomic DNA is cell-free DNA.

42. The method of claim 36, wherein the maternal sample is maternal plasma or serum.

43. The method of claim 36, wherein the fetal aneuploidy is an aneuploidy selected from the group consisting of chromosome 13, chromosome 18, chromosome 21, chromosome X and chromosome Y.

44. The method of claim 36, wherein the informative single nucleotide polymorphisms are tag single nucleotide polymorphisms.

45. A method for determining fetal fraction in a maternal sample, wherein the method comprises:
a. obtaining a mixture of fetal and maternal genomic DNA from said maternal sample;
b. conducting massively parallel DNA sequencing of random DNA fragments from the mixture of fetal and maternal genomic DNA of step a) to determine the sequence of said DNA fragments;
c. identifying nucleic acids corresponding to a plurality of single nucleotide polymorphisms by alignment of the sequenced DNA fragments to a reference;
d. determining the relative frequency of the sequenced single nucleotide polymorphisms;
e. comparing the determined relative frequencies of the single nucleotide polymorphisms to a fetal proportion reference; and
e. estimating the fetal fraction of the maternal sample based on the comparison of the determined relative frequencies of the single nucleotide polymorphisms to the fetal proportion reference.

46. The method of claim 45, wherein the sequence of the DNA fragments is from about 15 bp to about 150 bp in length.

47. The method of claim 46, wherein the sequence of the DNA fragments is from about 25 bp to about 100 bp in length.

48. The method of claim 45, wherein the genomic DNA is cell-free DNA.

49. The method of claim 48, wherein the maternal sample is maternal plasma or serum.

50. The method of claim 45, further comprising determining the number of single nucleotide polymorphisms necessary for a statistically significant estimation of fetal fraction in the maternal sample.
